(19) United States
(12) Patent Application Publication
(10) Pub. No.: US 2019/0029651 A1
(43) Pub. Date: Jan. 31, 2019

(54) CLUTTER FILTERS FOR STRAIN AND OTHER ULTRASONIC DEFORMATION IMAGING

(71) Applicant: KONINKLIJKE PHILIPS N.V., EINDHOVEN (NL)

(72) Inventors: Abhay Vijay Patil, Andover, MA (US); Francois Guy Gerard Marie Vignon, Andover, MA (US); Sheng-Wen Huang, Ossining, NY (US); Scott William Dianis, Andover, MA (US); Karl Erhard Thiele, Andover, MA (US)

(21) Appl. No.: 16/077,558

(22) PCT Filed: Feb. 27, 2017
(86) PCT No.: PCT/EP2017/054436

§ 371 Date: Aug. 13, 2018

Related U.S. Application Data
(60) Provisional application No. 62/300,233, filed on Feb. 26, 2016.

(51) Int. Cl. A61B 8/08; A61B 8/00

(52) U.S. Cl. CPC A61B 8/5276; A61B 8/485; A61B 8/463; A61B 8/14; A61B 8/5223; A61B 8/5246; A61B 8/0883

(57) ABSTRACT

An ultrasonic diagnostic imaging system and method filter the data of successive ultrasonic images of moving tissue. At least two successive images are clutter filtered to remove clutter in the image data from stationary objects. The successive images and the filtered successive images are both used to produce displacement images of frame to frame tissue motion, and a quality metric is produced of the quality of the estimated displacement. The displacement image with the best quality metric is selected for further processing such as the production of strain images. The selected displacement image may further be spatially filtered to reduce boundary artifacts.

This invention relates to medical diagnostic ultrasound systems and, in particular, to ultrasound systems which assess tissue stiffness and function through the assessment of tissue deformation.

### BACKGROUND

Elastography is the assessment of the elastic properties of tissue in the body. It has been found that the stiffness of tissue in the body can give an indication of whether the tissue may be malignant or benign. The female breast, for instance, can contain a variety of different lumps, cysts, and other growths, some of which may be malignant and some of which may be benign. To spare the patient from needless biopsies, and perform them when needed, ultrasound is frequently used to assess tissue characteristics to determine whether to biopsy suspect tissue. Elastography can be performed to determine whether the breast contains softer or harder (stiffer) regions. Since stiffer tissue correlates more greatly with malignant masses, the identification of regions of stiffer tissue can indicate a need to make a definitive diagnosis by biopsy.

A problem posed by elastography is the need to measure quantifiable properties of tissue noninvasively within the body. This means that the properties of the target tissue cannot be measured directly at the site of the tissue, but only through measurements made at the surface of the body through intervening tissues. Accordingly it is desirable to simplify the problem and make certain approximations and assumptions that will lead to valid data and analyses. One set of assumptions that is frequently made is that the tissue being examined is homogeneous and isotropic. These assumptions enable certain property of materials equations to be applied to the problem, Poisson's ratio and Young's modulus. Poisson's ratio is the ratio, when a sample is stretched or compressed in a given direction, of the expansion or contraction (strain) normal to the stretching or compressing force, to the expansion or contraction axially in the direction of the force. A related measure is Young's modulus, which is a measure of stiffness, and is defined as the ratio of the uniaxial stress (pressure) applied to a sample over the resulting uniaxial strain (deformation). However, the stress component at target tissue is generally unknown and difficult to measure noninvasively.

The analysis of tissue deformation (strain) is also applied in cardiology to assess the viability of myocardial tissue. Healthy heart tissue contracts strongly during the systolic phase of the heart cycle, whereas tissue which has suffered necrosis due to an infarct is incapable of such deformation. Necrosed tissue will be dragged along by surrounding healthy myocardial tissue and will not itself aid in cardiac contractility. Thus, strain imaging is often used to detect and quantify regions of myocardial tissue which have been damaged by infarction.

In order to assess cardiac strain, it is necessary to track the motion of specific regions or points of the myocardium through the heart cycle. This tracking is generally performed by following specific tissue or image characteristics of the myocardium such as the speckle characteristic of localized tissue resulting from the coherent nature of ultrasonic imaging. However, reflections and reverberations from the chest wall, ribs and lungs can give rise to clutter in the echo data used for imaging. Such clutter can manifest itself as static or slow-moving features that are superimposed over and interfere with the desired echo signals from the myocardial tissue. Clutter negatively affects image quality and hinders segmentation and automatic tracking algorithms used for the imaging of cardiac deformation.

One common way to eliminate clutter has been routinely used in ultrasonic colorflow imaging over the past thirty years, the wall or clutter filter. These clutter filters rely upon the distinctly different velocities of the motion of flowing blood and other bodily fluids as compared with the much slower motion of tissue. The clutter or wall filter for blood flow imaging is a high pass filter which passes the higher velocity blood flow signals while filtering out the generally much stronger signals returned from slowly moving tissue such as the walls of the heart chambers and blood vessels. However, the clutter filters used to image blood flow cannot be implemented for cardiac tissue deformation imaging for two reasons. First, the ensemble length for cardiac tissue Doppler is very low due to the low motional velocities of tissue, and there are insufficient signal samples to adequately operate a multi-tap filter such as those used in colorflow. Secondly, the tissue of interest in cardiac deformation imaging is generally static or slow-moving for a significant portion of the cardiac cycle, which results in the cancellation by a wall filter of the signal of interest for the imaging of tissue deformation. Elimination of the signal of interest renders such a filter inapposite for the imaging or analysis of tissue motion.

### SUMMARY

Accordingly it is desirable to be able to eliminate clutter in signal data from moving tissue so as to produce more accurate analytical results such as strain estimation in myocardial tissue.

In accordance with the principles of the present invention a clutter filtering technique and apparatus are described which can remove clutter from ultrasonic signals returned from slowly moving tissue.

According to certain aspect, the present invention provides an ultrasonic diagnostic imaging system which reduces clutter in images of moving tissue. The system includes a source of successive frames of ultrasonic image data of moving tissue, a clutter filter configured to filter the successive frames to reduce clutter, thereby generating clutter-reduced successive frames of the moving tissue; and a tissue displacement estimates selector. The tissue displacement estimates selector is configured to
estimate a first displacement between successive frames of ultrasonic image data of moving tissue;
estimate a second displacement between the clutter-reduced successive frames;
generate an indication of the quality of first and second estimated displacements; select the estimated displacement which exhibits the best quality; and output a displacement image characterized by the selected tissue displacement.

According to certain aspects, the inventive technique and apparatus receive successive frames of ultrasonic image data of the target anatomy, preferably at a relatively high frame rate of acquisition. The data can be operated upon as r.f. data or as detected (e.g., envelope detected) data. Successive frames of data are clutter filtered in slow-time, that is, as a function of the acquisition frame rate, to eliminate signal data from stationary objects. Since the subject signals are from slowly moving tissue, shorter length filters (fewer taps) are generally employed. Frame to frame tissue displacement is then estimated for both the filtered and unfiltered data. The displacement estimate judged to be of the highest quality, e.g., the best frame to frame correlation, is then selected as the displacement estimate to be used for imaging. Finally, discontinuities in the image data due to the use of both filtered and unfiltered data can be removed by a spatial or spatial/temporal filter. The moving tissue data thus processed is then used for analysis such as for the production of strain images as described below.

### IN THE DRAWINGS

FIG. 1 illustrates in block diagram form an ultrasonic diagnostic imaging system constructed in accordance with the principles of the present invention.

FIG. 2 illustrates a corner-turning data table which can be used in an implementation of the present invention.

FIG. 3 illustrates a two-tap FIR filter suitable for use in an implementation of the present invention.

FIG. 4 is a flowchart of an implementation of the clutter filtering technique of the present invention.

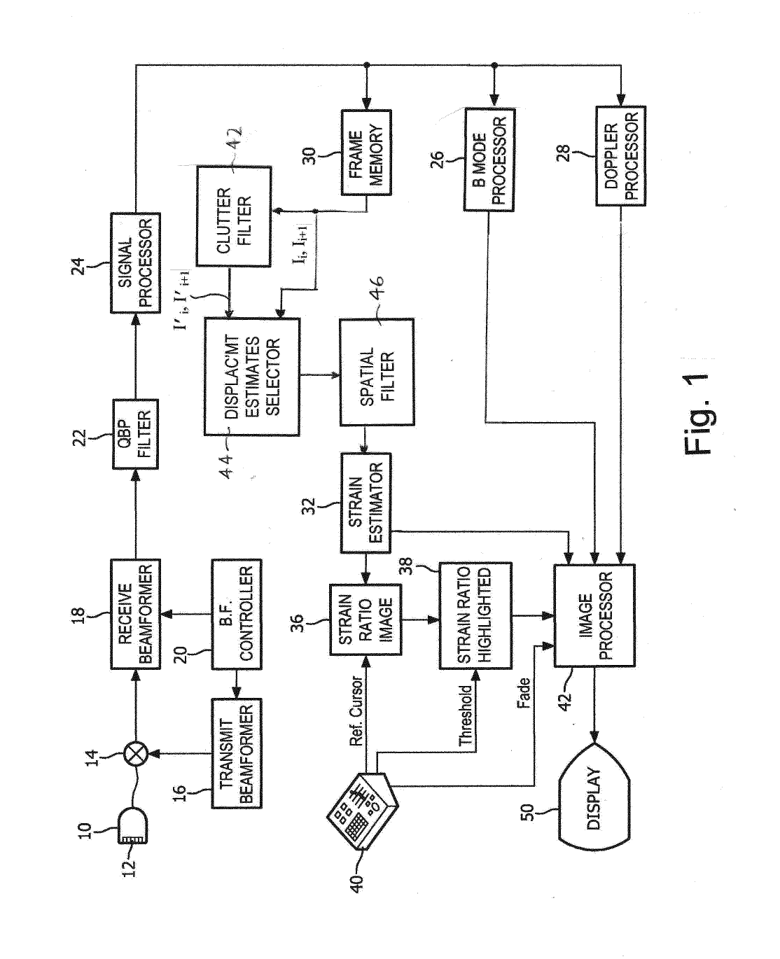

Referring first to FIG. 1, an ultrasound system constructed in accordance with the principles of the present invention is shown in block diagram form. An ultrasound probe 10 has an array transducer 12 which transmits ultrasound waves to and receives echoes from a region of the body. The array transducer can be a one-dimensional array of transducer elements or a two-dimensional array of transducer elements for scanning a two dimensional image field or a three dimensional image field in the body. The elements of the array transducer are driven by a transmit beamformer 16 which controls the steering, focusing and penetration of transmit beams from the array. A receive beamformer 18 receives echoes from the transducer elements and combines them to form coherent echo signals from points in the image field. The transmit and receive beamformers are coupled to the transducer array elements by transmit/receive switches 14 which protect sensitive receive circuitry during transmission. A beamformer controller 20 synchronizes and controls the operation of the beamformers.

The received echo signals are demodulated into quadrature (I and Q) samples by a quadrature bandpass (QBP) filter 22. The QBP filter can also provide band limiting and bandpass filtering of the received signals. The received signals may then undergo further signal processing such as harmonic separation and frequency compounding by a signal processor 24. The processed echo signals are stored in a frame memory 30 where the echo data is stored as a corner turning memory for use in a clutter filter of the present invention as discussed below. The echo signals are also applied to a B mode processor 26 which performs amplitude detection of the echo signals by the equation (I²+Q²)^{1/2} for formation of a B mode image, and to a Doppler processor 28 for Doppler shift detection of flow motion at points in the image field. The outputs of the B mode processor 26 and the Doppler processor 28 are coupled to an image processor 42. The image processor processes the image data from these sources, e.g., by scan conversion to a desired image format, image overlay, etc., and produces an image for display on a display 50.

In accordance with the principles of the present invention the frame memory 30 stores successive time-separated samplings of the image field on a spatial basis. For cardiac imaging the frame rate of the image acquisition is preferably at least 150 frames per second to avoid aliasing as per the Nyquist criterion. A higher frame rate of at least 800 Hz can also be used for aliasing phase shift (aka Doppler) estimators to estimate the motion. For imaging other anatomy with different motional characteristics other frame rates may be used. The image data of these frames is filtered by clutter filter 42 which filters the data as a function of the frame acquisition rate: samples (pixels) of the image field acquired from the same spatial location in different images are high-pass filtered by the clutter filter 42. The clutter filter has a cutoff frequency around DC so as to reject static tissue motion. Short-length FIR filters have been found effective for this filtering of tissue motion, such as a two-tap filter with tap weights of [+1] and [−1], or a three-tap filter with weights of [+1], [−2] and [+1]. The number of taps used depends on the expected range of motion of the tissue, with faster motion requiring shorter filters, and the bandwidth around DC that one wishes to reject. In some embodiments, more than one clutter filter can also be used.

The filtered frame data and the unfiltered frame data from the frame memory 30 are applied to a displacement estimates selector 44. The displacement estimates selector estimates the tissue displacement indicated for the frame-to-frame data for both the clutter-filtered and the unfiltered image data. There can be one displacement estimate for the unfiltered data and one displacement estimate per clutter filter used to filter the data. As described further herein, only one clutter filter is described for simplicity, but the extension to using several clutter filters would be readily understood by one of ordinary skill in the art. The inputs to the displacement estimates selector in the drawing show the input of the data of one image I; and a subsequent image Iᵢ₊₁ for the unfiltered images and the input of the corresponding filtered images I′₁ and I′ᵢ₊₁. The algorithm used to estimate the frame-to-frame displacements also produces a quality metric for the displacement estimates. This metric may take the form of the local normalized cross correlation coefficient from a correlation of the successive pixel data. Another possibility is to calculate the local normalized cross-correlation coefficient between frames aligned according to the displacement estimates. Other metrics which can be used include the non-normalized correlation coefficient, sum of absolute differences, or sum of square differences. The displacement estimates selector 44 then selects a displacement estimate to be used for the final image, either that from the clutter-filtered data, that from the unfiltered data, or a combination thereof. The selection can be made based on the quality metric, the more favorable metric indicating the displacement to be chosen. This selection is independently made for each pixel of the final image. For example, if at one pixel location the filtered data yields a higher cross correlation than that of the non-filtered data, the final displacement estimate is the one that was estimated from the filtered data.

Since this selection is made for each pixel in the image field, it is to be expected that there will be some regions of the final image where the displacement of the filtered data is used, and other regions where the displacement of the unfiltered data is used. It is then possible that spatial discontinuities may be present where one such region transitions into another. These discontinuities may be eliminated by filtering the final image data with a spatial filter 46, which may also be implemented as a spatial/temporal filter. Four-point or eight-point spatial filtering may be employed, as well as other spatial filtering or averaging techniques.

The images produced by the spatial filter 46 are used for the calculation of strain by a strain estimator 32, operating on the frame-to-frame displacement of particles in the image field; strain is calculated as a spatial derivative of displacement. Strain may be calculated from radio frequency (r.f.) or baseband I and Q data, and may also be calculated from amplitude-detected (B mode) or tissue Doppler data. Any form of strain calculation such as strain, the ratio of lateral to axial strain, and strain velocity estimation may be employed. For instance, the echoes received at a common point in consecutive frames may be correlated to estimate displacement at the point. If no motion is present at the point, the echoes from consecutive frames will be the same. If motion is present, the echoes will be different and the motion vector indicates the displacement. U.S. Pat. No. 6,558,324 (Von Behren et al.) describes both amplitude and phase sensitive techniques for estimating strain and employs speckle tracking for strain estimation through block matching and correlation. U.S. Pat. No. 6,099,471 (Torp et al.) describes the estimation of strain velocity calculated as a gradient of tissue velocity. U.S. Pat. No. 5,800,356 (Criton et al.) describes the use of the Doppler vector to select points for strain estimation in the direction of the tissue motion. Preferably a phase-sensitive technique is used since, as recognized by Von Behren et al., r.f. data will typically yield the most accurate estimates of strain. Another reason for the preference of strain estimation with phase-sensitive techniques is that the slight motion produced by physiological activity and even from the small, virtually imperceptible motion occurring while holding a probe against the body can be sensed and used to estimate strain by the strain estimator 32.

The strain estimator 32 produces an estimated strain value at each point (pixel location) in the image field, and these values are spatially organized by the strain estimator as a strain image of the image field. The strain image is coupled to the image processor 42, as are the outputs of the B mode processor 26 and the Doppler processor 28, and the strain image is displayed on display 50.

In accordance with a further aspect of the present invention the strain image is also used to produce a strain ratio image at 36. The strain ratio image is produced by dividing each strain value of the strain image by a strain value for normal tissue. This value may be provided automatically as by averaging or taking the mean or median value of a plurality of strain values, such as the strain values in a region in a corner of an image (on the assumption that the user will position suspect target anatomy in the center of the image.) In an implementation of the present invention the strain value for normal tissue is taken from an indication of normal tissue in an image which is indicated by a user. For this purpose the strain ratio image is responsive to a reference cursor from a control panel 40 manipulated by the user to indicate a point or region of normal tissue in an image. Each strain value in the strain image is divided by a strain value of normal tissue to produce a strain ratio image at 36.

To better delineate suspect tissue region in the strain ratio image, the user may manipulate a control of the control panel 40 to set a threshold or range of values against which the strain ratio values are compared. Strain ratio values which exceed the threshold or the range of values are uniquely highlighted in the strain ratio image as by displaying them with unique colors or brightnesses. For example the user can set the threshold at 5, and all points in the strain ratio display with a value of 5 or greater, indicative of high stiffness, can be displayed in a bright red color. The user can quickly spot suspect regions in the image from the distinctive bright red color in the strain ratio image.

In accordance with yet another aspect of the present invention the user can manipulate a fade control on the control panel 40 which is coupled to the image processor 42. When the image processor displays a strain ratio image overlaying the corresponding portion of a B mode image, the fade control enables the user to adjust the relative transparency of the B mode and strain ratio images. The user can fade the strain ratio image to be completely transparent to see the corresponding structural image alone, or can fade the B mode tissue image to see only the strain ratio image, or an intermediate transparency setting for the two overlaid images.

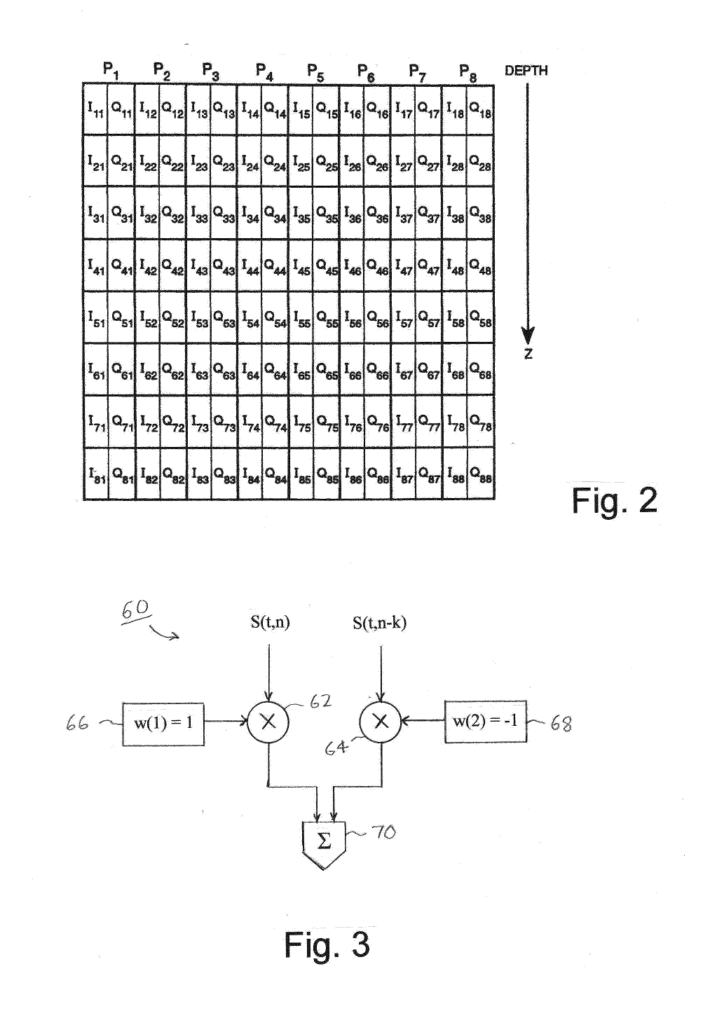

FIG. 2 illustrates a useful way of organizing the image data in frame memory 30, which in this example is that of a corner turning memory. A corner turning memory is typically used to store echo data used for Doppler processing as it organizes the data in rows of common spatial locations, across which temporally different samples are stored. Thus, each row of data comprises an ensemble of data for a spatial location (pixel) which may be Fourier-processed to yield a Doppler estimate. The vertical direction (depth z) is the “fast time” direction in which echo signals received by sampling from along one transmit-receive A-line are stored. The fast time sampling rate in a typical ultrasound system is on the order of many megaHertz. In FIG. 2, the first subscript of each I,Q value is the fast time subscript, indicating the sequence in which the I,Q sample values are received along the line. The horizontal axis of the data is the “slow time” axis indicating the pulses Pₙ which generate the echoes from transmitting at successive times along the same spatial A-line, which is typically denominated in Hertz (frames per second.) The second subscript of each I,Q value indicates the slow time sequence of each I,Q sample of a row of data, each row containing the data from temporal sampling of the same pixel location in the image field. Thus, the data table of FIG. 2 illustrates the quadrature data acquired from eight samples in depth along an A-line of an image field (an actual ultrasound system may sample a line scores or hundreds of times,) and the A-line has been sampled eight times in succession by transmitting eight temporally successive pulses P₁-P₈ along the A-line.

Since the table of FIG. 2 only represents the data acquired by temporally sampling a single A-line of an image, the frame memory 30 will contain other data tables from the acquisition of echo signal data from all A-lines of an image. A typical two-dimensional image may be formed by 128adjacent A-lines, for example, and thus the frame memory for such an image will contain 128 tables of data like the one shown in FIG. 2. The frame memory 30 is generally an addressable digital random access memory, for instance.

FIG. 3 illustrates a two-tap FIR filter 60 suitable for use in clutter filter 42. In this example a signal from a spatial location in one image pixel value, S(t,n), is applied to one tap of the filter and the pixel value of the same spatial location in a temporally different image, S(t,n-k) is applied to the other tap of the filter. In this notation a signal S is denominated by a fast time acquisition time t and a slow time of acquisition n or n-k, respectively. Thus, the pixel values at a common spatial location of two successively acquired images are applied to the taps of the filter and weighted by weights of +1 and −1, respectively, as stated for one of the example filters described above. The filter 60 thus exhibits a slow time time constant. Multipliers 62 and 64 multiply the image values by the weights and the weighted image values are summed by a summer 70 to produce the filtered output value of the clutter filter. As mentioned above, different length filters can be used; a three-tap implementation would have a third tap with a third multiplier for a third temporally different pixel value, which is summed with the others by the summer 70.

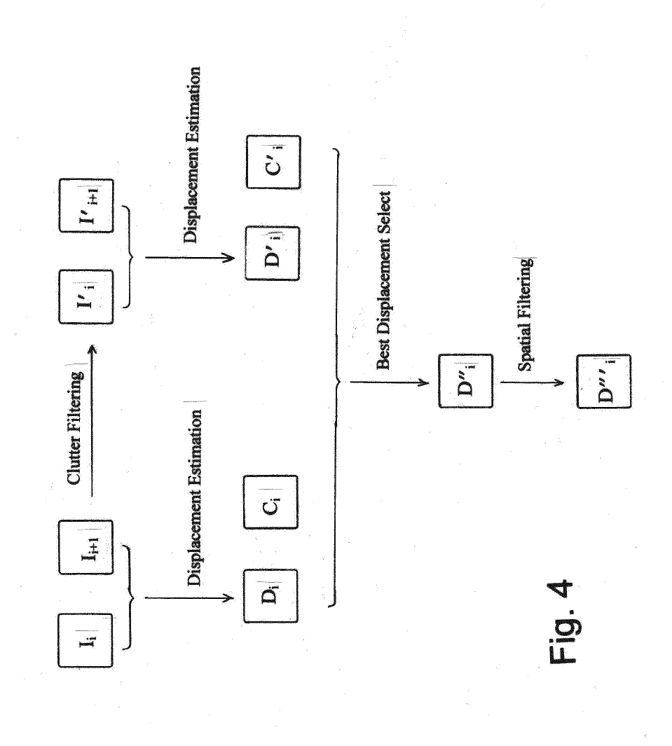

FIG. 4 is a flowchart illustration of the operation of frame memory 30, clutter filter 42, displacement estimates selector 44, and spatial filter 46 of FIG. 1 in accordance with the principles of the present invention. Blocks Iᵢ and Iᵢ₊₁ represent the frame data stored in frame memory 30 from the acquisition of successive images of the same image field of a body. To the right at the top of the drawing are blocks I′ᵢ and I′ᵢ₊₁ representing the same images after clutter filtering. In the next row of the drawing is block Dᵢ, representing the pixel-by-pixel displacement estimates caused by a motional difference between the original two images Iᵢ and Iᵢ₊₁. Next to the displacement data block is a quality metric block Cᵢ containing the cross-correlation coefficients of the cross-correlation of the two images. To the right are corresponding blocks D′ᵢ and C′ᵢ representing the displacement estimates and cross-correlation coefficients for the clutter-filtered image data. The displacement estimates selector then selects for output the displacement data with the best quality metric C, on a pixel-by-pixel basis, which is shown as block D″ᵢ. The displacement data image is then smoothed by spatial filtering to produce the final image D′″ᵢ for strain estimation processing.

In a constructed implementation of the present invention, the operation of the clutter filter 42, the displacement estimates selector 44, and the spatial filter are all performed by digital software running on a microprocessor and programmed to execute algorithms that effect the desired processing of the acquired echo signal data stored in the frame memory 30. Typical algorithms which may be employed are the following. The local signal of one A-line for multiple frames can be described as

s(t,n)

where t indexes fast-time and n indexes slow-time (i.e., frame index). Tissue displacements from frame to frame can be locally estimated by computing the normalized cross-correlation function of multiple lags:

$${ρ}_{ap} ( τ ) = \frac{{\sum }_{t} s ( t , n ) {s ( t + τ , n + 1 )}^{*}}{\sqrt{{\sum }_{t} {\vert s ( t , n ) \vert}^{2} {\sum }_{t} {\vert s ( t + τ , n + 1 ) \vert}^{2}}}$$

The displacement τₐₚ that maximizes ρₐₚ(ρ) is the estimate of the interframe displacement (the subscript ‘ap’ stands for “all-pass”). In this example, the high-pass-filtered version of s(t, n) is first calculated (in slow-time) according to

$${s}_{hp} ( t , n ) = {\sum }_{k} w ( k ) s ( t , n - k )$$

where the coefficients w(k) are those of a high-pass filter. The simplest embodiment is with w(1)=1 and w(2)=−1, amounting to subtracting successive frames. The objective is to eliminate stationary signal components from the input signal.

One can compute the cross-correlation function to estimate inter-frame displacements from sₕₚ(t, n):

$${ρ}_{ap} ( τ ) = \frac{{\sum }_{t} {s}_{hp} ( t , n ) {{s}_{hp} ( t + τ , n + 1 )}^{*}}{\sqrt{{\sum }_{t} {\vert {s}_{hp} ( t , n ) \vert}^{2} {\sum }_{t} {\vert {s}_{hp} ( t + τ , n + 1 ) \vert}^{2}}}$$

The displacement τₕₚ that maximizes ρₕₚ(τ) is another estimate of the interframe displacement (the subscript ‘hp’ stands for “high-pass”). In some cases ρₐₚ may be a better estimate of the real displacement (i.e., when there is little inter-frame displacement and the high-pass filtering operation destroys all useful signals); in other cases ρₕₚ may be a better estimate (i.e., when there is a presence of stationary tissue clutter that can be eliminated by the high-pass filtering operation). The objective is to find, for each pixel, which of these two estimates is the best. This is simply done by comparing the corresponding values of the cross-correlation functions and picking the estimate that gives the highest value, i.e.:
Pick τₐₚ if ρₐₚ(τₐₚ)≥ρₕₚ(Σₕₚ) or
Pick τₐₚ if ρₐₚ(τₐₚ)<ρₕₚ(Σₕₚ).

More generally, the final estimated displacement can be a blend of the two estimates according to

$${τ}_{final} = \frac{f ( {ρ}_{h p} ( {τ}_{h p} ) , {ρ}_{ap} ( {τ}_{ap} ) ) {τ}_{hp} + g ( {ρ}_{hp} ( {τ}_{hp} ) , {ρ}_{ap} ( {τ}_{ap} ) ) {τ}_{ap}}{f ( {ρ}_{h p} ( {τ}_{h p} ) , {ρ}_{ap} ( {τ}_{ap} ) ) + g ( {ρ}_{hp} ( {τ}_{hp} ) , {ρ}_{ap} ( {τ}_{ap} ) )}$$

where f and g are blending functions of the correlation coefficients.

At a boundary a blend or average of the two values may be the desired result. The process is repeated for all image pixels and frames at which an estimate of the inter-frame displacement is desired. The final output is a 2D or 3D image of displacement, τ(x, y, z, t). The map of displacement data is finally smoothed by applying a low-pass spatial (and possibly temporal) filter:

$${τ}_{final} ( x , y , z , t ) = {{\sum }_{x}}^{′} {{\sum }_{y}}^{′} {{\sum }_{z}}^{′} {{\sum }_{t}}^{′} w ( x - {x}^{′} , y - {y}^{′} , z - {z}^{′} , t - {t}^{′} ) {τ}_{final} ( {x}^{′} , {y}^{′} , {z}^{′} , {t}^{′} )$$

where w(x, y, z, t) is a slowly varying function in space and time.

Note that many formulations can be used to estimate the displacements τₐₚ and Tₕₚ, for example the non-normalized cross-correlation of the sum of square differences are commonly used functions to maximize to find a displacement:

Non-normalized cross-correlations:

$$c ( τ ) = {\sum }_{t} s ( t , n ) {s ( t + τ , n + 1 )}^{*}$$

Sum of square differences:

$$d ( τ ) = {\sum }_{t} {\vert s ( t , n ) - s ( t + τ , n + 1 ) \vert}^{2}$$

Or a simple Doppler phase shift estimation can be performed:

τ˜angle(Σₜs(t, n)s(t,n+1)*)

The sum can also be extended over slow-time and over the other spatial dimensions for added robustness, e.g.,

$$d ( τ ) = {\sum }_{t} {\sum }_{n} {\vert s ( t , n ) - s ( t + τ , n + 1 ) \vert}^{2}$$

Weights could also be added for robustness as in

$$d ( τ ) = {\sum }_{t} {\sum }_{n} w ( t , n ) {\vert s ( t , n ) - s ( t + τ , n + 1 ) \vert}^{2}$$

However the normalized cross-correlation at the lag that maximizes the chosen metric is still preferred when choosing to pick one or the other estimate for the inter-frame displacement.

The ultrasound system components and processors of FIG. 1 may be implemented utilizing any combination of dedicated hardware boards, DSPs, microprocessors, etc. and software programs stored on a system disk drive or solid state memory. Alternatively, the functionality and components of the system may be implemented utilizing an off-the-shelf PC with a single microprocessor or multiple microprocessors, with the functional operations distributed between the processors. As a further option, the functionality of FIG. 1 may be implemented utilizing a hybrid configuration in which certain modular functions are performed utilizing dedicated hardware, while the remaining modular functions are performed utilizing an off-the shelf PC, software and the like. An example of this configuration is a probe containing the transducer array and microbeamformer to produce beamformed echo signals, which are then further processed to produce images entirely by software programs of a tablet computer, on which the final images are displayed. The Philips Healthcare Visiq ultrasound system is an example of such a system implementation, in which all of the ultrasound system functionality after beamforming is performed by software executed by the tablet microprocessor. The various functions of the blocks shown in FIG. 1 also may be implemented as software modules within a processing unit.

It should be noted that the various embodiments described above and illustrated by the exemplary ultrasound system of FIG. 1 may be implemented in hardware, software or a combination thereof. The various embodiments and/or components, for example, the modules, or components and controllers therein, also may be implemented as part of one or more computers or microprocessors. The computer or processor may include a computing device, an input device, a display unit and an interface, for example, for accessing the Internet. The computer or processor may include a microprocessor. The microprocessor may be connected to a communication bus, for example, to access a PACS system. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, solid-state thumb drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

Furthermore, the limitations of the following claims are not written in means-plus-function format and are not intended to be interpreted based on 35U.S.C. 112, sixth paragraph, unless and until such claim limitations expressly use the phrase “means for” followed by a statement of function devoid of further structure.

1. An ultrasonic diagnostic imaging system comprising:
a source of successive frames of ultrasonic image data of moving tissue;
a clutter filter configured to filter the successive frames to produce successive frames of filtered data of the moving tissue; and
a tissue displacement estimates selector configured to:
estimate first displacements between pixels of the successive frames of ultrasonic image data of the moving tissue to produce a first displacement image and respective first quality metrics for the first displacements;
estimate second displacements between corresponding pixels of the successive frames of the filtered data to produce a second displacement image and respective second quality metrics for the second displacements;
generate a third displacement image comprising estimated displacements selected from the first and second displacements based on a comparison between the respective first and second quality metrics.

2. The ultrasonic diagnostic imaging system of claim 1, further comprising a spatio-temporal filter configured to receive the third displacement image and to spatially filter the third displacement image.

3. The ultrasonic diagnostic imaging system of claim 1, wherein the clutter filter comprises a high pass filter.

4. The ultrasonic diagnostic imaging system of claim 3, wherein the high pass filter is finite impulse response filter.

5. The ultrasonic diagnostic imaging system of claim 3, wherein the high pass filter further is a two-tap filter.

6. The ultrasonic diagnostic imaging system of claim 3, wherein the high pass filter is a three-tap filter.

7. The ultrasonic diagnostic imaging system of claim 3, wherein;
the high pass filter is configured to filter the successive frames of ultrasonic image data in a slow time time.

8. The ultrasonic diagnostic imaging system of claim 1, further comprising a strain estimator configured to generate a strain image comprising strain values estimated from the third displacement image;
a display operable to display the strain image.

9. The ultrasonic diagnostic imaging system of claim 8, wherein the strain estimator further generates the estimated strain values as local estimates of frame-to-frame image deformation.

10. The ultrasonic diagnostic imaging system of claim 9, wherein the frame-to-frame image deformation is produced by physiological motion of a subject being imaged.

11. The ultrasonic diagnostic imaging system of claim 9, further comprising a reference indicator to indicate a location of reference tissue in the image field; and
a strain ratio processor configured to produce a strain ratio image comprising ratios of strain values at points in the image field to a strain value of the reference tissue.

12. A method for filtering time-sequential ultrasonic images of moving tissue, the method comprising:
acquiring a plurality of time-sequential ultrasonic images of moving tissue;
clutter filtering the plurality time-sequential ultrasonic images to produce a plurality of respective filtered ultrasonic images of the,moving tissue;
estimating first displacements between corresponding pixels of the plurality of time-sequential ultrasonic images and producing a first displacement image and respective first quality metrics for the first displacements;
estimating second displacements between corresponding pixels of the plurality of respective filtered time-sequential ultrasonic images and producing a second displacement image and respective second quality metrics for the second displacement; and
generating a third displacement image comprising estimated displacements by selecting, pixel by pixel, from the first second displacements based on a comparison between the respective first and second quality metrics.

13. The method of claim 12, further comprising spatio-temporal filtering the third displacement image.

14. (canceled)

15. The method of claim 12, further comprising producing a strain image utilizing the third displacement image.
